(19) United States
(12) Patent Application Publication
(10) Pub. No.: US 2023/0410673 A1
(43) Pub. Date: Dec. 21, 2023

(54) SYSTEM AND METHOD FOR PROVIDING CUSTOMIZED SAFETY EDUCATION AND EXPERIENCE SERVICE FOR DISASTERS BASED ON VIRTUAL REALITY

(71) Applicant: METAEDUSYS CO., LTD., Andong-si (KR)

(72) Inventor: Jonghee BAE, Andong-si (KR)

(21) Appl. No.: 18/209,572

(22) Filed: Jun. 14, 2023

(30) Foreign Application Priority Data
Jun. 16, 2022 (KR) 10-2022-0073520
Dec. 9, 2022 (KR) 10-2022-0171160

(51) Int. Cl. G09B 5/06; A61B 5/16

(52) U.S. Cl. CPC G09B 5/065; A61B 5/165

(57) ABSTRACT

Provided are a system and method for providing a customized safety education and experience service for disasters on the basis of virtual reality. The method performed by a server includes receiving experience request information from an experiencer who enters a virtual experience space, generating a customized experience scenario using the experience request information, and executing an experience service using the customized experience scenario.

### CROSS-REFERENCE TO RELATED APPLICATION

This application claims priority to and the benefit of Korean Patent Application No. 2022-0073520, filed on Jun. 16, 2022, and Korean Patent Application No. 2022-0171160, filed on Dec. 9, 2022, the disclosure of which is incorporated herein by reference in its entirety.

### BACKGROUND OF THE INVENTION

### Field of the Disclosure

The present invention relates to a system and method for providing a customized safety education and experience service for disasters on the basis of virtual reality (VR), and more particularly to, a system and method for providing a customized safety education and experience service for disasters on the basis of VR which enable a user to experience a disaster through a direct stimulus on the skin using a thermoelectric element for adjusting a feeling of cold or warm.

### Description of the Related Art

In general, virtual reality (VR) is a technology for generating an artificial environment that may interact with a user through his or her sense of sight, hearing, touch, etc. and enabling the user to feel as if an environment that does not physically exist actually existed.

VR is also called cyberspace, artificial reality, virtual world, synthetic environment, artificial environment, etc.

Such VR is used in various fields, such as medicine, architecture, games, aviation, nuclear power, etc., and particularly used for the purpose of training for disasters that are difficult to experience in everyday reality.

Recently, interest in school safety education has grown due to a rapid increase in various disasters, and school safety education has been under examination. However, overall inadequacies in safety education were confirmed due to unsystematized safety education and safety education by non-experts. Accordingly, the need for an omnidirectional safety education control tower to play a central role in school safety education has increased.

In particular, the need for safety education to prepare for disasters including various natural disasters, such as earthquakes, typhoons, heavy rains, tsunamis, lighting strikes, landslides, etc., safety-related accidents, such as gas accidents, fire accidents, electrical accidents, traffic accidents, etc., and industrial accidents such as falls occurring at industrial sites and the like is rising.

However, there are few facilities in which people can experience types of natural disasters, and a facility in which all types of disasters can be experienced has not been developed yet. Also, facilities in which people can experience a specific disaster are very lacking.

The above description of the background art is only for the purpose of improving the understanding of the background of the present invention and should not be taken as an acknowledgement that the description corresponds to related art already known to those of ordinary skill in the art.

### PRIOR ART DOCUMENT

### Patent Document

Korean Patent No. 10-0616333

### SUMMARY

The present invention is directed to providing a system and method for providing a customized safety education and experience service for disasters on the basis of virtual reality (VR).

Objects of the present invention are not limited to that described above, and other objects which have not been described will be clearly understood by those of ordinary skill in the art from the following descriptions.

According to an aspect of the present invention, there is provided a method of providing a customized safety education and experience service for disasters on the basis of VR, by a server, the method including receiving experience request information from an experiencer who enters a virtual experience space, generating a customized experience scenario using the experience request information, and executing an experience service using the customized experience scenario. The executing of the experience service includes receiving surrounding information detected in accordance with a customized VR video corresponding to the virtual experience space, analyzing the surrounding information to determine whether there is a disaster, analyzing the surrounding information and current location information in accordance with a result of determining whether there is a disaster to determine whether the experiencer is in danger, receiving current physical and emotion information of the experiencer in accordance with a result of determining whether the experiencer is in danger, generating changed physical and emotion information by analyzing basic physical and emotion information and the current physical and emotion information to, and executing a countermeasure scenario generated in accordance with the changed physical and emotion information on the basis of standard data.

The determination of whether there is a disaster may include determining that there is a disaster when surrounding cold or warm feeling information is larger than a preset reference value on the basis of the surrounding information, and determining that there is no disaster when the surrounding cold or warm feeling information is smaller than the preset reference value on the basis of the surrounding information.

The determination of whether the experiencer is in danger may include determining that the experiencer is in danger when it is determined that there is a disaster and current cold or warm feeling information is larger than the preset reference value on the basis of the surrounding information and the current location information, and determining that the experiencer is not in danger when it is determined that the current cold or warm feeling information is smaller than the preset reference value on the basis of the surrounding information and the current location information.

The generating of the changed physical and emotion information may include, when the current physical and emotion information is larger than a preset reference value, detecting a physical change to generate bio-detection information and detecting an emotion change to generate emotion detection information, and comparing basic physical information in the basic physical and emotion information with the bio-detection information and comparing basic emotion information in the basic physical and emotion information with the emotion detection information to generate the changed physical and emotion information.

The executing of the countermeasure scenario may include analyzing the changed physical and emotion information on the basis of the standard data, and when the analyzed changed physical and emotion information is larger than the preset reference value, determining that the experiencer undergoes a great emotion change and physical change and generating a countermeasure scenario for finishing experience or analyzing the changed physical and emotion information on the basis of the standard data, and when the analyzed changed physical and emotion information is smaller than the preset reference value, determining that the experiencer undergoes a great emotion change and physical change and generating a countermeasure scenario for continuing the experience service.

Depending on a situation, the countermeasure scenario may include at least one of a countermeasure scenario for a case in which there is a disaster during the executing of the experience service, a countermeasure scenario for a case in which there is no disaster, a countermeasure scenario for a case in which the experiencer is in great danger after a disaster occurs, a countermeasure scenario for a case in which the experiencer is not in great danger after a disaster occurs, and a countermeasure scenario for an emotion change of the experiencer when the experiencer is in great danger after a disaster occurs.

According to another aspect of the present invention, there is provided a method of providing a customized safety education and experience service for disasters on the basis of VR, the method including receiving experience request information from an experiencer who enters a virtual experience space, generating a customized experience scenario using the experience request information, and executing an experience service using the customized experience scenario. The generating of the customized experience scenario includes analyzing the experience request information to extract basic physical and emotion information of the experiencer and generating the customized experience scenario using the basic physical and emotion information on the basis of standard data.

The executing of the experience service may include outputting a customized VR video corresponding to the customized experience scenario, receiving surrounding information detected in accordance with the customized VR video corresponding to the virtual experience space, when the surrounding information is larger than a preset reference value, determining that there is a disaster and receiving current location information of the experiencer, analyzing the surrounding information and the current location information to determine whether the experiencer is in danger and receiving current physical and emotion information of the experiencer when it is determined that the experiencer is in danger, analyzing the current physical and emotion information to generate a countermeasure scenario, and finishing the experience service on the basis of the countermeasure scenario.

The generation of the countermeasure scenario may include, when the current physical and emotion information is larger than the preset reference value, analyzing the current physical and emotion information on the basis of the basic physical and emotion information to generate changed physical and emotion information and generating the countermeasure scenario matching the changed physical and emotion information on the basis of the standard data.

The generation of the changed physical and emotion information may include, when the experiencer equipped with an experience terminal which comes into direct contact with a body part and has at least one sensor is exposed to a preset condition or higher in VR, generating bio-detection information for controlling a temperature of a thermoelectric element in accordance with an exposure condition, detecting an emotion change of the experiencer in the VR using the bio-detection information to generate emotion detection information, and comparing basic physical information in the basic physical and emotion information with the bio-detection information and comparing basic emotion information in the basic physical and emotion information with the emotion detection information to generate the changed physical and emotion information.

The generation of the emotion detection information may include receiving a brainwave signal in real time from a brainwave measurement sensor provided in the experience terminal, analyzing the brainwave signal through a main control unit (MCU) to calculate an amount of stress of each process, and generating the emotion detection information.

The method may further include, before the executing of the experience service, transmitting the customized VR video in accordance with the VR information on the basis of standard data not in a solo mode but in a collaboration mode in which at least two people experience VR.

The method may further include, after the finishing of the experience service, assessing quality of the experience on the basis of a result and a score of the experience service to generate result data and generating a customized experience scenario using the experience request information of the experiencer on the basis of the result data.

The method may further include generating a countermeasure scenario for dealing with an unexpected situation corresponding to a disaster during the execution of the experience service.

According to another aspect of the present invention, there is provided a system for providing a customized safety education and experience service for disasters on the basis of VR, the system including a management server configured to provide a customized safety education and experience service to an experiencer equipped with an experience terminal which comes into direct contact with a body part and has at least one sensor in VR. The management server receives experience request information from the experiencer who enters a virtual experience space, generates a customized experience scenario using the experience request information, and executes an experience service using the customized experience scenario. Also, the management server extracts basic physical and emotion information of the experiencer by analyzing the experience request information and generates the customized experience scenario using the basic physical and emotion information on the basis of standard data.

According to another aspect of the present invention, there is provided a program stored in a computer-readable recording medium to perform the method of providing a customized safety education and experience service for disasters on the basis of VR in combination with a computer which is hardware.

Other details of the present invention are included in the detailed description and the accompanying drawings.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a conceptual diagram illustrating a system for providing a customized safety education and experience service for disasters on the basis of virtual reality (VR) according to an exemplary embodiment of the present invention;

FIG. 2 is a diagram illustrating a detailed configuration of the system for providing a customized safety education and experience service for disasters on the basis of VR shown in FIG. 1;

FIG. 3 is a set of views illustrating a detailed configuration of an experience terminal shown in FIGS. 1 and 2;

FIG. 4 is a diagram illustrating a detailed configuration of a controller shown in FIG. 3;

FIG. 5 is a flowchart illustrating a method of providing a customized safety education and experience service for disasters on the basis of VR according to an exemplary embodiment of the present invention;

FIG. 6 is a flowchart illustrating an operation of generating a customized experience scenario shown in FIG. 5;

FIG. 7 is a flowchart illustrating an operation of executing an experience service shown in FIG. 5 according to an exemplary embodiment of the present invention;

FIG. 8 is a flowchart illustrating the operation of executing an experience service shown in FIG. 5 according to another exemplary embodiment of the present invention; and

FIG. 9 is a flowchart illustrating a method of providing a customized safety education and experience service for disasters on the basis of VR according to another exemplary embodiment of the present invention.

### DETAILED DESCRIPTION OF EXEMPLARY EMBODIMENTS

Advantages and features of the present invention and a method of achieving them will become apparent from embodiments which will be described in detail below with reference to the accompanying drawings. However, the present invention is not limited to the embodiments disclosed below and may be implemented in various forms. The embodiments are only provided to make the disclosure of the present invention complete and fully convey the scope of the present invention to those skilled in the technical field to which the present invention pertains. The present invention is only defined by the scope of the claims.

Terminology used herein is for the purpose of describing embodiments only and is not intended to limit the present invention. As used herein, the singular forms include the plural forms as well unless the context clearly indicates otherwise. The terms “comprises” and/or “comprising” do not preclude the presence or addition of one or more components other than stated components. Throughout the specification, like reference numerals refer to like components, and “and/or” includes any one or all possible combinations of stated components. Although “first,” “second,” etc. are used to describe various components, the components are not limited by the terms. These terms are used to distinguish one component from other components. Accordingly, it is apparent that a first component described below may be a second component within the technical spirit of the present invention.

Unless otherwise defined, all terms (including technical and scientific terms) may have meanings generally understood by those skilled in the technical field to which the present invention pertains. Also, unless clearly defined, all terms defined in generally used dictionaries are not to be ideally or excessively interpreted.

Hereinafter, exemplary embodiments of the present invention will be described in detail with reference to the accompanying drawings.

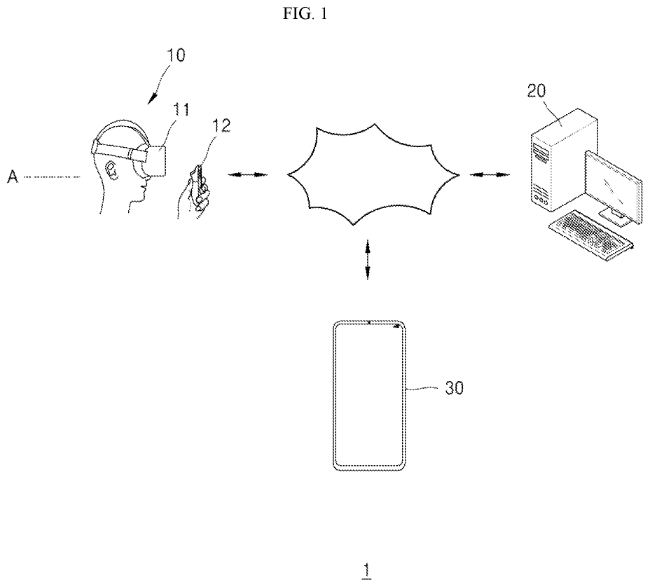

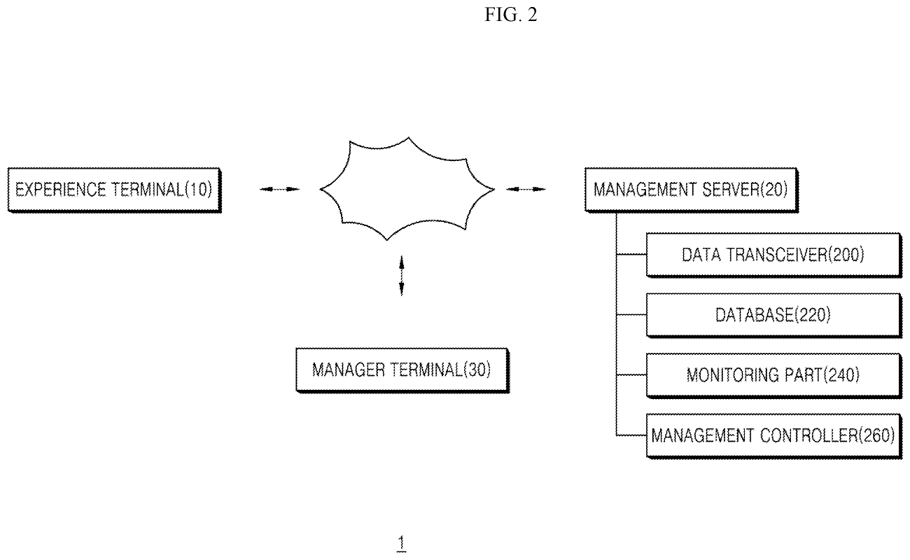

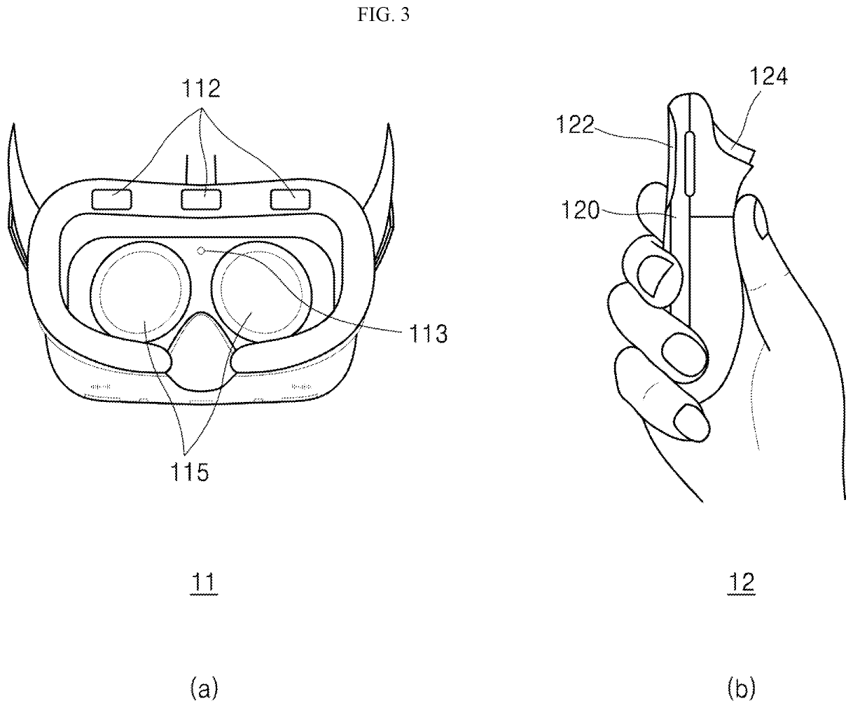

FIG. 1 is a conceptual diagram illustrating a system for providing a customized safety education and experience service for disasters on the basis of virtual reality (VR) according to an exemplary embodiment of the present invention, and FIG. 2 is a diagram illustrating a detailed configuration of the system for providing a customized safety education and experience service for disasters on the basis of VR shown in FIG. 1. FIG. 3 is a set of views illustrating a detailed configuration of an experience terminal shown in FIGS. 1 and 2, and FIG. 4 is a diagram illustrating a detailed configuration of a controller shown in FIG. 3.

First, when an experiencer A equipped with an experience terminal 10 enters a virtual experience space, a system 1 for providing a customized safety education and experience service for disasters on the basis of VR may provide a service by visually, auditorily, and tactilely implementing situations of disasters including various natural disasters, such as earthquakes, typhoons, heavy rains, tsunamis, lighting strikes, landslides, etc., safety-related accidents, such as gas accidents, fire accidents, electrical accidents, traffic accidents, etc., industrial accidents, such as falls occurring at industrial sites and the like, etc. but is not limited thereto.

Also, the virtual experience space is a space that may be used in accordance with a selection of a user (hereinafter “experiencer”) and/or a paid or free service, and may be used as each individual's private experience space. Alternatively, the virtual experience space may be used by a plurality of experiencers.

According to an exemplary embodiment, the experience service may be used through membership subscription of an experiencer.

As shown in FIGS. 1 and 2, the system 1 for providing a customized safety education and experience service for disasters on the basis of VR according to the exemplary embodiment of the present invention may include the experience terminal 10, a management server 20, and a manager terminal 30.

Here, the experience terminal 10, the management server 20, and the manager terminal 30 may be synchronized in real time using a wireless communication network and transmit and receive data. The wireless communication network may support various long-range communication methods. For example, various communication methods, such as wireless local area network (WLAN), Digital Living Network Alliance (DLNA), Wireless Broadband (WiBro), World Interoperability for Microwave Access (WiMAX), Global System for Mobile Communications (GSM), Code Division Multiple Access (CDMA), CDMA2000, Enhanced Voice-Data Optimized or Enhanced Voice-Data Only (EV-DO), Wideband CDMA (WCDMA), High Speed Downlink Packet Access (HSDPA), High Speed Uplink Packet Access (HSUPA), Institute of Electrical and Electronic Engineers (IEEE) 802.16, Long Term Evolution (LTE), LTE Advanced (LTEA), Wireless Mobile Broadband Service (WMBS), Bluetooth Low Energy (BLE), ZigBee, radio frequency (RF), Long Range (LoRa), etc., may be applied. However, applicable communication methods are not limited thereto, and various widely known wireless communication methods or mobile communication methods may be applied.

The experience terminal 10 may perform an experience service through communication with the management server 20.

Specifically, the experience terminal 10 is a wearable device that is directly worn on a body part for a VR experience. In the present disclosure, the experience terminal 10 may operate using an application program or application, and the application program may be downloaded from an external server or the management server 20 through wireless communication.

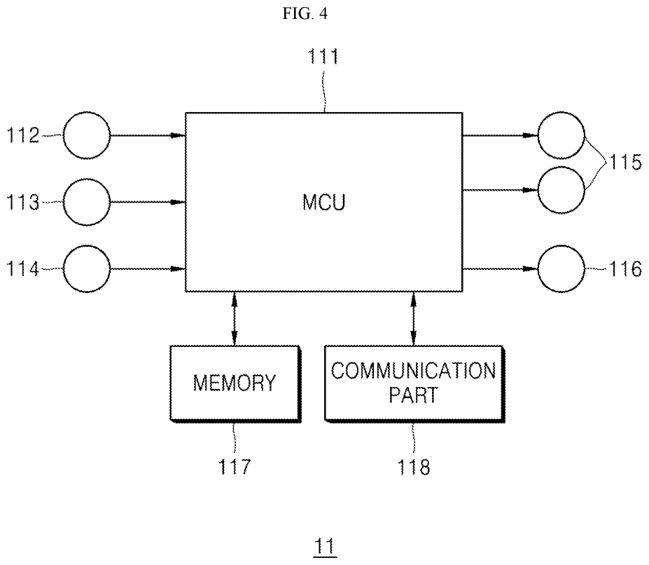

As shown in FIGS. 3 and 4, the experience terminal 10 may include a VR head mounted display (TIMID) 11 which is worn on the head of the experiencer A and a controller 12 which is wirelessly connected to the VR HMD 11.

More specifically, referring to FIGS. 3A and 4, the VR HMD 11 may include a brainwave measurement sensor 112 which comes into direct contact with the forehead of the experiencer A to measure brainwaves, a camera 113 which detects a movement direction and angle of the eyes of the experiencer A, a motion sensor 114 which detects a movement direction and angle of the head of the experiencer A, left and right lenses 115 which provide an image or video screen to the experiencer A, a sound output part 116 which outputs a voice message or a sound signal, a memory 117 with software which receives a voice, an image, or a video for dealing with a disaster and a control signal for selecting or executing the voice, the image, or the video and provides various pieces of content, a wireless communication part 118 which communicates with the controller 12 or an external device, and a main control unit (MCU) 111 which executes an experience service stored in the memory 117, calculates a stress status of the experiencer every time period using the brainwaves input from the brainwave measurement sensor 112, moves the screen in accordance with the eyes or movement direction of the experiencer A input through the camera 113 or the motion sensor 114, and receives the control signal of the controller 12 through the wireless communication part 118 to display a corresponding screen.

According to the present exemplary embodiment, the MCU 111 may calculate an emotion status of the experiencer A using the brainwaves input from the brainwave measurement sensor 112.

Here, the memory 117 may be a memory chip having a secure digital (SD) card form. Accordingly, the memory 117 may be attached or detached through an insertion hole provided on one external side surface of the VR HMD 11 so that memory chips of various content can be inserted.

Also, the memory 117 may store a plurality of application programs or applications run on the VR HMD 11 and data and instructions for operations of an electroencephalogram (EEG) device 10. At least some of the application programs may be downloaded from an external server through wireless communication.

The controller 12 may be wirelessly connected to the VR HMD 11 and may include a thermoelectric element 120, a touchpad 122, and an execution button 124.

Specifically, as shown in FIG. 3B, the thermoelectric element 120 may be embedded in a middle portion of the controller 12 which comes into contact with a hand of the experiencer A who holds the controller 12, and may provide a stimulus to the experiencer A.

During a virtual experience, the thermoelectric element 120 may detect a high or low temperature in accordance with a situation of a disaster, receive cold or warm feeling information, and deliver a feeling of high-temperature heat or low-temperature cold to the experiencer A in real time.

For example, a virtual experience environment may be a situation in which a type of disaster that breaks out is a fire. In this case, when the experiencer A is exposed to the fire, smoke, and hot air for a certain time, the thermoelectric element 120 may increase in temperature and emit heat, thereby providing bio-detection information having a feeling of high-temperature heat to the experiencer A.

In other words, when a body part of the experiencer A is exposed to the fire, the smoke, and the hot air for the certain time or more, the thermoelectric element 120 corresponding to the body part in the exposure direction may detect the exposure and emit heat to the exposed body part of the experiencer A, thereby delivering a feeling of high-temperature heat.

Alternatively, a virtual experience environment may be a situation in which a type of disaster is drifting on the sea. In this case, when the experiencer A is exposed to the sea water and cold air for a certain time, the thermoelectric element 120 may decrease in temperature and absorb heat, thereby providing bio-detection information having a feeling of low-temperature cold to the experiencer A.

In other words, when a body part of the experiencer A is exposed to the sea water and the cold air for the certain time or more, the thermoelectric element 120 corresponding to the body part in the exposure direction may detect the exposure and absorb heat from the exposed body part of the experiencer A, thereby delivering a feeling of low-temperature cold.

According to an exemplary embodiment, the controller 12 may include a temperature sensor (not shown) that is included in a contact surface which comes into contact with the skin of the experiencer A to detect a temperature change between the skin of the experiencer A and the thermoelectric element 120.

For example, an increase in temperature between the thermoelectric element 120 and the skin may be detected so that the skin of the experiencer A can be prevented from being damaged by an excessive temperature increase. In this way, the experiencer A can safely use the controller 12.

The thermoelectric element 120 receives cold or warm feeling information in accordance with the situation of a disaster and delivers a feeling of high-temperature heat or a feeling of low-temperature cold to the experiencer A in real time in accordance with the situation. Accordingly, the experiencer A can experience a realistic disaster and deal with the disaster, rescue people, or escape from the disaster properly and safely without panicking.

While the touchpad 122 is held and pressed by the hand of the experiencer A, the experiencer A may be allowed to select and move to a desired location in the virtual experience space.

The execution button 124 is a button for performing a set function when it is pressed by the experiencer A, and may be replaced with a general remote control.

Meanwhile, according to an exemplary embodiment, the experience terminal 10 may include power supply (not shown) that may receive external power or internal power and supply the power to each component included in the EEG device 10. The power supply may include a battery (not shown), and the battery level may be visually checked. The battery may be connected to a 220 V commercial power source, a laptop computer, or a desktop computer through a Universal Serial Bus (USB) cable for charging. Also, the battery may be a cell phone battery. A 3.7 V lithium-ion battery which is the most economical and efficient secondary battery may be used to allow charging with a cell phone battery charger. Alternatively, the battery may be an embedded battery or a battery in a removable form.

Before the experiencer A enters the virtual experience space, the experience terminal 10 having the above configuration may generate experience request information of the experiencer A to execute the experience service.

Here, the experience request information may include basic information, such as an age, a sex, etc. of the experiencer A, additional information, such as an occupation, a hobby, inclination, etc., bio-information, such as a height, a weight, a musculoskeletal mass, a body fat content, a heart rate, a blood pressure, an oxygen saturation, etc., emotion information, such as happiness, neutrality, anger, disgust, fear, sadness, surprise, etc. of the experiencer A, and medical information such as a plurality of pieces of electronic medical record (EMR) information measured at a plurality of medical institutions. However, the experience request information is not limited thereto and may include other information such as the priority of experience, the preference of experience, etc. regarding types of disasters.

Here, the inclination may include, but is not limited to, information, such as rational, sensitive, emotional, conservative, progressive, passive, negative, positive, etc., and the EMR information may include, but is not limited to, a treatment record, dosage information, bio-parameter information, a drug response, and a family history of the experiencer A, an expert's opinion on the experiencer A, etc.

According to an exemplary embodiment, the experience request information may be input in advance by the experiencer A through membership subscription before the experience service is executed. Alternatively, the experience request information may be input for a one-time use without membership subscription.

Also, the experience terminal 10 may execute the experience service and provide a customized experience scenario corresponding to the experience request information to the experiencer A.

Specifically, the experience terminal 10 may visually, auditorily, and tactilely provide a situational countermeasure scenario which is in accordance with a disaster and included in the customized experience scenario corresponding to the experience request information using a first-person 360° VR video.

According to the present embodiment, countermeasure scenarios may include a countermeasure scenario for a case in which there is a disaster during execution of the experience service, a countermeasure scenario for a case in which there is no disaster, a countermeasure scenario for a case in which the experiencer A is in great danger after a disaster occurs, and a countermeasure scenario for a case in which the experiencer A is not in great danger after a disaster occurs. However, countermeasure scenarios are not limited thereto and may include a countermeasure scenario for an emotion change of the experiencer A when the experiencer A is in great danger after a disaster occurs.

Also, after the experience service is finished, the experiencer A may generate and transmit feedback information regarding the experience service.

The feedback information may include information, such as satisfaction, accuracy, reliability, etc., on the experience service. Here, the feedback information may be generated and transmitted after the experience service is used, but may also be transmitted before or during use of the experience service.

According to an exemplary embodiment, the experiencer A may receive advertising information or event information including information on a discount or event for the experience service from the management server 20, but the present invention is not limited to this case.

According to an exemplary embodiment, after finishing the experience, the experiencer A may receive result data that is generated by assessing the quality of the experience on the basis of a result and a score of the experience service.

The experiencer A may require re-experience in accordance with the result data when the quality of the experience is low. In other words, due to the re-experience, the experiencer A can deal with a disaster, rescue people, or escape from the disaster properly and safely without panicking.

According to an exemplary embodiment, when standard data is received from the management server 20, the experience terminal 10 may generate a customized experience scenario corresponding to the experience request information on the basis of the standard data.

As described above, the experiencer A may enter a virtual experience space using the experience terminal 10 and receive an experience service through a customized experience scenario generated in accordance with experience request information on the basis of standard data. Accordingly, the experience terminal 10 visually, auditorily, and tactilely provides the experience service using objective information and subjective information of the experiencer A who requests the experience service, thereby respecting the diversity of users who receive experience services and enabling the experiencer A to accurately recognize the dangerousness of a disaster in accordance with a situation and properly deal with various actual disasters. Therefore, it is possible to improve convenience and reliability.

Also, the experience terminal 10 includes the thermoelectric element 120 that receives cold or warm feeling information in accordance with the situation of a disaster and delivers a feeling of heat or cold in real time, thereby giving a direct stimulus to the skin of the experiencer A. Therefore, the experiencer A can be further immersed in VR through a feeling on his or her skin.

The management server 20 may include a data transceiver 200, a database 220, a monitoring part 240, and a management controller 260.

The data transceiver 200 may transmit or receive data to or from the experience terminal 10 or the manager terminal 30.

The database 220 may store the data that is transmitted or received to or from the experience terminal 10 or the manager terminal 30 through the wireless communication network.

The database 220 may store data for supporting various functions of the management server 20. The database 220 may store a plurality of application programs or applications run on the management server 20 and data and instructions for operations of the management server 20. At least some of the application programs may be downloaded from an external server through wireless communication.

The monitoring part 240 may monitor an operating state of the experience terminal 10 caused by manipulation of the experiencer A, an operating state of the management server 20, data transmitted and received to or from the experience terminal 10 or the manager terminal 30, etc. in real time through a screen. In other words, the state of using the experience terminal 10 is checked in real time, which facilitates use of the experience terminal 10 by the experiencer A. Accordingly, the user may have confidence.

When experience request information for a disaster is received from the experience terminal 10, the management controller 260 may generate a customized experience scenario for the disaster on the basis of the standard data, receive result information corresponding to the generated customized experience scenario from the experience terminal 10, and generate result data.

Specifically, the management controller 260 may analyze the experience request information on the basis of the standard data and generate a customized experience scenario for each disaster situation.

For example, when experience request information is received from the experiencer A who enters the virtual experience space, the management controller 260 may generate a customized experience scenario including a situational countermeasure scenario suitable for a current state of the experiencer A in consideration of surrounding information and current location information on the basis of the standard data.

Here, the experience request information may include basic information, such as the age, the sex, etc. of the experiencer A, additional information, such as the occupation, the hobby, the inclination, etc., bio-information, such as the height, the weight, the musculoskeletal mass, the body fat content, the heart rate, the blood pressure, the oxygen saturation, etc., emotion information, such as happiness, neutrality, anger, disgust, fear, sadness, surprise, etc. of the experiencer A, and medical information such as the plurality of pieces of EMR information measured at the plurality of medical institutions. However, the experience request information is not limited thereto and may include other information such as the priority of experience, the preference of experience, etc. regarding types of disasters. Here, the basic information, the bio-information, and the medical information may be determined as objective information, and the additional information, the emotion information, and the other information may be determined as subjective information.

The inclination information may include, but is not limited to, information, such as rational, sensitive, emotional, conservative, progressive, passive, negative, positive, etc., and the EMR information may include, but is not limited to, the treatment record, the dosage information, the bio-parameter information, the drug response, and the family history of the experiencer A, the expert's opinion on the experiencer A, etc.

According to the present embodiment, countermeasure scenarios may include a countermeasure scenario for a case in which there is a disaster during execution of the experience service, a countermeasure scenario for a case in which there is no disaster, a countermeasure scenario for a case in which the experiencer A is in great danger after a disaster occurs, and a countermeasure scenario for a case in which the experiencer A is not in great danger after a disaster occurs. However, countermeasure scenarios are not limited thereto and may include a countermeasure scenario for an emotion change of the experiencer A when the experiencer A is in great danger after a disaster occurs.

For example, the management controller 260 may analyze the surrounding information of the experiencer A that is input in real time in the virtual experience space. When surrounding cold or warm feeling information included in the surrounding information is larger than a preset reference value, the management controller 260 may determine that there is a disaster and generate a countermeasure scenario for a case in which a disaster occurs.

Here, the cold or warm feeling information is information directly transmitted to the experiencer A as a feeling of heat or cold by the thermoelectric element 120 receiving a high or low temperature around the experiencer A and may include surrounding cold or warm feeling information and current cold or warm feeling information.

In other words, when fire, smoke, and hot air around the experiencer A last three minutes or more within a radius of 100 mm in accordance with surrounding information received in the virtual experience space, the thermoelectric element 120 may receive the surrounding cold or warm feeling information and determine that there is a disaster of a fire, and the management controller 260 may generate a countermeasure scenario for dealing with the fire situation, rescuing people, or escaping from the fire situation.

Meanwhile, when it is determined that there is a disaster due to a value of the surrounding information larger than the preset reference value, the management controller 260 may receive current location information of the experiencer A from a customized VR video and analyze the surrounding information and the current location information on the basis of the standard data. When the experiencer A is determined to be in great danger, the management controller 260 may generate a countermeasure scenario for a case in which the experiencer A is in great danger.

For example, when fire, smoke, and hot air around the experiencer A last three minutes or more within a radius of 100 mm in accordance with surrounding information received in the virtual experience space, the thermoelectric element 120 may receive cold or warm feeling information and determine that there is a disaster of a fire. In this case, when it is determined that the experiencer A is in an area of the disaster using the current location information of the experiencer A and the surrounding information, the management controller 260 may generate a countermeasure scenario for a case in which the experiencer A is in great danger due to current cold or warm feeling information larger than the present reference value.

According to an exemplary embodiment, when a disaster occurs, the management controller 260 may determine whether the experiencer A is in danger by analyzing surrounding information and current location information. When it is determined that the experiencer A is in danger, the management controller 260 may compare and analyze basic physical and emotion information of the experiencer A extracted from the experience request information and current physical and emotion information received from the experiencer A and generate a countermeasure scenario for a case in which the experiencer A is in great danger.

Specifically, when the current physical and emotion information is larger than the preset reference value, the management controller 260 may generate changed physical and emotion information by analyzing the current physical and emotion information on the basis of the basic physical and emotion information and generate a countermeasure scenario matching the changed physical and emotion information on the basis of the standard data.

For example, when a body part of the experiencer A holding the controller 12 is exposed to danger in the virtual experience space at a preset condition or more, the management controller 260 may generate bio-detection information having a feeling of high-temperature heat or low-temperature cold received by the experiencer A in response to cold or warm feeling information received in accordance with an exposure condition, generate emotion detection information by detecting an emotion change of the experiencer A in the VR using the bio-detection information, and then generate changed physical and emotion information by comparing the basic physical information with the bio-detection information and comparing the basic emotion information with the emotion detection information. A countermeasure scenario may be generated using the generated changed physical and emotion information.

In other words, when fire, smoke, and hot air or sea water and cold air around the experiencer A last three minutes or more within a radius of 100 mm in accordance with surrounding information received in the virtual experience space, the thermoelectric element 120 may receive surrounding cold or warm feeling information and increase the temperature to emit heat and deliver a feeling of heat to the experiencer A or decrease the temperature to absorb heat and deliver a feeling of cold to the experiencer A. In this case, the management controller 260 may generate bio-detection information in accordance with the cold or warm feeling information, generate emotion detection information by detecting an emotion change of the experiencer A in the VR in accordance with the generated bio-detection information, generate changed physical and emotion information by comparing and analyzing the basic physical and emotion information and current physical and emotion information, and then generate a countermeasure scenario matching the generated changed physical and emotion information on the basis of the standard data.

According to an exemplary embodiment, the changed physical and emotion information may be acquired by comparing only the bio-detection information with the basic physical information or comparing only the emotion detection information with the basic emotion information.

According to an exemplary embodiment, the management controller 260 may analyze the changed physical and emotion information on the basis of the standard data, and when the changed physical and emotion information is larger than the reference value, may determine that the experiencer A undergoes a big emotion change and physical change and generate a countermeasure scenario for finishing the experience.

Alternatively, the management controller 260 may analyze the changed physical and emotion information on the basis of the standard data, and when the changed physical and emotion information is smaller than the reference value, may determine that the experiencer A undergoes a small emotion change and physical change and generate a countermeasure scenario for continuing the experience service.

Here, the emotion detection information may be acquired by receiving an emotion state of the experiencer A in danger in real time through brainwave signals from the brainwave measurement sensor 112 provided in the experience terminal 10 and may represent an emotion state changed from the basic emotion information. However, the emotion detection information is not limited thereto.

According to an exemplary embodiment, the management controller 260 may receive brainwave signals in real time from the brainwave measurement sensor 112 provided in the experience terminal 10, analyze the brainwave signals through the MCU 111, and calculate the amount of stress in each process, thereby generating emotion detection information.

Also, the management controller 260 may analyze the current physical and emotion information on the basis of the standard data, and when the current physical and emotion information is smaller than the reference value, may determine that the experiencer A undergoes a small emotion change and physical change and generate a countermeasure scenario for continuing the experience service.

When it is determined that there is a disaster due to a value of the surrounding information larger than the preset reference value, the management controller 260 may receive current location information of the experiencer A from the customized VR video and analyze the surrounding information and the current location information on the basis of the standard data. When the experiencer A is not determined to be in great danger, the management controller 260 may generate a countermeasure scenario for a case in which the experiencer A is not in great danger.

For example, fire, smoke, and hot air around the experiencer A may last three minutes or more within a radius of 100 mm in accordance with surrounding information received in the virtual experience space, and thus a disaster of a fire may occur. In this case, when it is determined that the experiencer A is not in an area of the disaster using the current location information and the surrounding information, the management controller 260 may generate a countermeasure scenario for a case in which the experiencer A is not in great danger.

Alternatively, the management controller 260 may analyze the surrounding information of the experiencer A which is input in real time in the virtual experience space, and when the surrounding information is smaller than the reference value, may determine that there is no disaster and generate a countermeasure scenario for a case in which there is no disaster.

In other words, when fire, smoke, and hot air around the experiencer A do not last three minutes or more within a radius of 100 mm in accordance with surrounding information received in the virtual experience space, the management controller 260 may determine that there is no disaster of a fire and generate a countermeasure scenario for a case in which there is no disaster.

Also, when the experiencer A equipped with the experience terminal 10 enters the virtual experience space, the management controller 260 may execute the experience service using a customized experience scenario.

Specifically, the management controller 260 may output a customized VR video corresponding to the customized experience scenario to the experiencer A and execute a situational countermeasure scenario in accordance with the virtual experience space and the customized VR video.

For example, the management controller 260 may receive surrounding information detected when the customized experience scenario is executed, determine whether there is a disaster by comparing the received surrounding information with the preset reference value, and then execute a situational countermeasure scenario corresponding to the determination result.

In other words, when surrounding cold or warm feeling information is larger than the preset reference value on the basis of the surrounding information, the management controller 260 may determine that there is a disaster, receive current location information of the experiencer A in the customized VR video, determine whether the experiencer A is in danger by analyzing the surrounding information and the current location information on the basis of the standard data, and then execute a situational countermeasure scenario corresponding to the determination result.

According to an exemplary embodiment, when there is a disaster, the management controller 260 may analyze the surrounding information and the current location information and determine whether the experiencer A is in danger. When it is determined that the experiencer A is in danger, the management controller 260 may receive current physical and emotion information of the experiencer A and execute a customized countermeasure scenario generated by comparing and analyzing the basic physical and emotion information and the received current physical and emotion information.

Alternatively, when the management controller 260 executes the experience service in accordance with a customized experience scenario, surrounding cold or warm feeling information may be smaller than the preset reference value on the basis of surrounding information detected in accordance with a customized VR video. In this case, the management controller 260 may determine that there is no disaster around the experiencer A and continuously output the customized VR video corresponding to the customized experience scenario.

For example, when fire, smoke, and hot air around the experiencer A do not last three minutes or more within a radius of 100 mm in accordance with surrounding information received in the virtual experience space, the thermoelectric element 120 does not receive surrounding cold or warm feeling information. Accordingly, it is determined that there is no disaster and the experiencer A is not in danger.

Also, when current cold or warm feeling information is larger than the preset reference value on the basis of surrounding information and current location information which are analyzed on the basis of a customized experience scenario, the management controller 260 may determine that the experiencer A is in danger and execute a situational countermeasure scenario corresponding to the determination result.

Meanwhile, the management controller 260 may determine whether the experiencer A is in danger using surrounding information and current location information which are analyzed on the basis of a customized experience scenario, and when it is determined that the experiencer A is in danger, may execute a countermeasure scenario generated by receiving and analyzing current physical and emotion information.

Specifically, when the current physical and emotion information is larger than the preset reference value, the management controller 260 may generate changed physical and emotion information by analyzing the current physical and emotion information on the basis of the basic physical and emotion information and execute a countermeasure scenario generated to match the changed physical and emotion information on the basis of the standard data.

For example, when fire, smoke, and hot air or sea water and cold air around the experiencer A last three minutes or more within a radius of 100 mm in accordance with surrounding information received in the virtual experience space, the thermoelectric element 120 may increase the temperature to emit heat and deliver a feeling of high-temperature heat to the experiencer A or may decrease the temperature to absorb heat and deliver a feeling of low-temperature cold to the experiencer A.

In other words, the management controller 260 may execute a countermeasure scenario corresponding to changed physical and emotion information which is generated by comparing and analyzing current physical and emotion information of the experiencer A who receives cold or warm feeling information and the basic physical and emotion information.

According to an exemplary embodiment, the management controller 260 may analyze the changed physical and emotion information on the basis of the standard data, and when the changed physical and emotion information is larger than the reference value, may determine that the experiencer A undergoes a big emotion change and physical change and execute a countermeasure scenario for finishing the experience.

Alternatively, the management controller 260 may analyze the changed physical and emotion information on the basis of the standard data, and when the changed physical and emotion information is smaller than the reference value, may determine that the experiencer A undergoes a small emotion change and physical change and generate a countermeasure scenario for continuing the experience service.

Meanwhile, the management controller 260 may analyze current physical and emotion information on the basis of the standard data, and when the current physical and emotion information is smaller than the reference value, may determine that the experiencer A undergoes a small emotion change and physical change and execute a countermeasure scenario for continuing the experience service.

Also, when current cold or warm feeling information is larger than the preset reference value on the basis of surrounding information and current location information which are analyzed on the basis of a customized experience scenario, the management controller 260 may determine that the experiencer A is not in danger and execute a situational countermeasure scenario for the determination result.

In a collaboration mode in which at least two or more people experience VR instead of a solo mode, the management controller 260 may transmit a customized VR video in accordance with the VR.

Specifically, in the collaboration mode, the management controller 260 may establish a communication link in advance so that experience terminals 10 carried by a plurality of experiencers A who enter a virtual experience space communicate with each other, and then may transmit a customized VR video in accordance with VR so that the same customized VR video is output to the experience terminals 10.

Here, the customized VR video may be a video corresponding to an average value of experience request information of the plurality of experiencers A who enter the virtual experience space on the basis of the standard data. However, the customized VR video is not limited thereto and may be a video corresponding to the collaboration mode on the basis of the standard mode.

According to an exemplary embodiment, after the experience is finished, the management controller 260 may assess the quality of the experience on the basis of a result and a score of the experience service and generate result data corresponding to the result information.

For example, when the quality of the experience is low and it is determined that re-experience is necessary in accordance with the result data of the experiencer A, the management controller 260 may generate a customized experience scenario using the result data of the experiencer A on the basis of the standard data. In other words, due to the re-experience, the experiencer A can deal with a disaster, rescue people, or escape from the disaster properly and safely without panicking.

Also, the management controller 260 may generate a countermeasure scenario corresponding to an unexpected situation which is a disaster during execution of the experience service.

For example, during the virtual experience, the management controller 260 may generate an unexpected situation of a disaster that is unpredictable to the experiencer A, thereby enabling the experiencer A to have various experiences of a disaster without being limited to a customized experience scenario generated on the basis of the experience request information of the experiencer A. Accordingly, safety education can be provided more usefully.

Also, the management controller 260 may generate a feedback control signal for improving the experience service in accordance with a feedback signal received from the experience terminal 10.

According to an exemplary embodiment, the management controller 260 may generate a feedback control signal including event information to increase use of the experience service by the experience terminal 10.

Also, the management controller 260 may generate the standard data by repeatedly learning big data using a deep learning technique or a machine learning technique.

Here, the standard data may be updated in real time in accordance with a customized experience scenario or result data.

According to an exemplary embodiment, the management controller 260 may update the standard data in real time in accordance with at least one of the feedback signal received from the experience terminal 10, the feedback control signal, and a countermeasure scenario.

The management server 20 may be implemented by a hardware circuit (e.g., a complementary metal-oxide semiconductor (CMOS)-based logic circuit), firmware, software, or a combination thereof. For example, the management server 20 may be implemented in the form of one of various electrical structures using transistors, logic gates, and electronic circuitry.

The manager terminal 30 is a terminal carried by a manager and may be synchronized in real time with the experience terminal 10 and the management server 20 using the wireless communication network to transmit and receive data. Here, the manager terminal 30 may transmit and receive data using an application program or an application.

The manager terminal 30 may generate a customized experience scenario using a customized experience scenario on the basis of the standard data or generate result data by assessing quality of the experience on the basis of a result and a score of the experience service after finishing the experience.

The manager terminal 30 may be one of various portable electronic communication devices that support communication with the experience terminal 10 and the management server 20. For example, the manager terminal 30 is a smart device and may be one of various portable terminals, such as a variety of Internet of Things (IoT) terminals including a smartphone, a personal digital assistant (PDA), a tablet, a wearable device, a smartwatch, smart glasses, a head mounted display (HMD), etc., but may also be a non-portable electronic communication device such as a desktop computer, a workstation computer, etc.

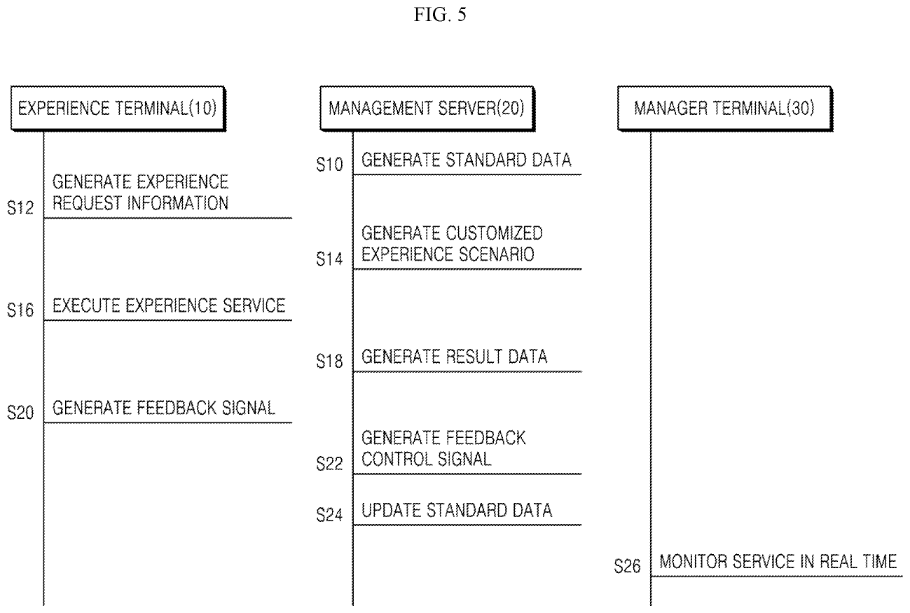

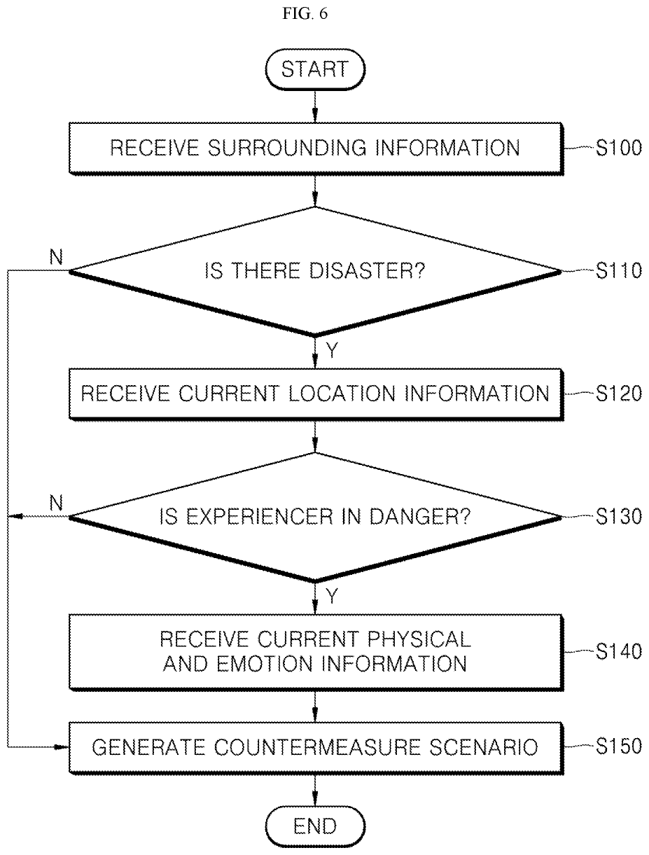

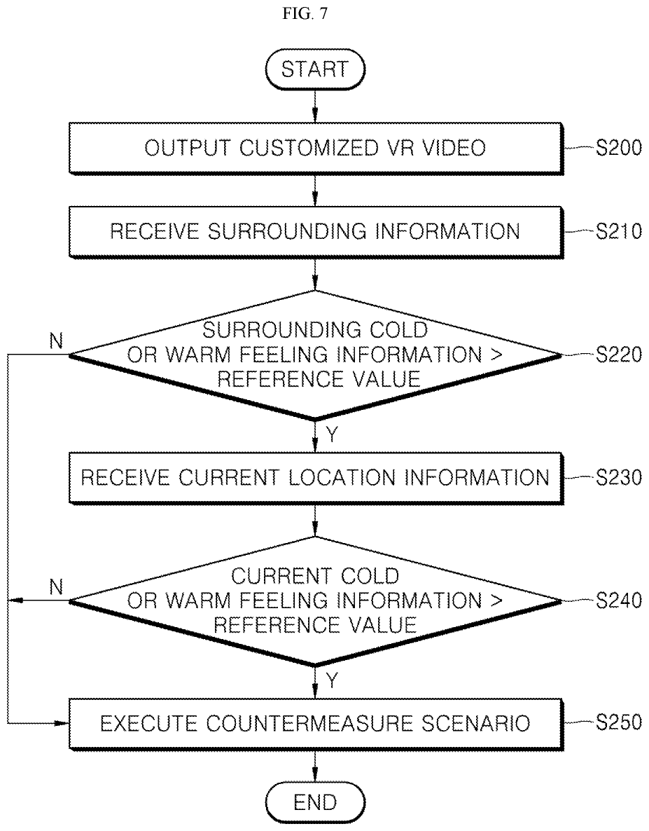

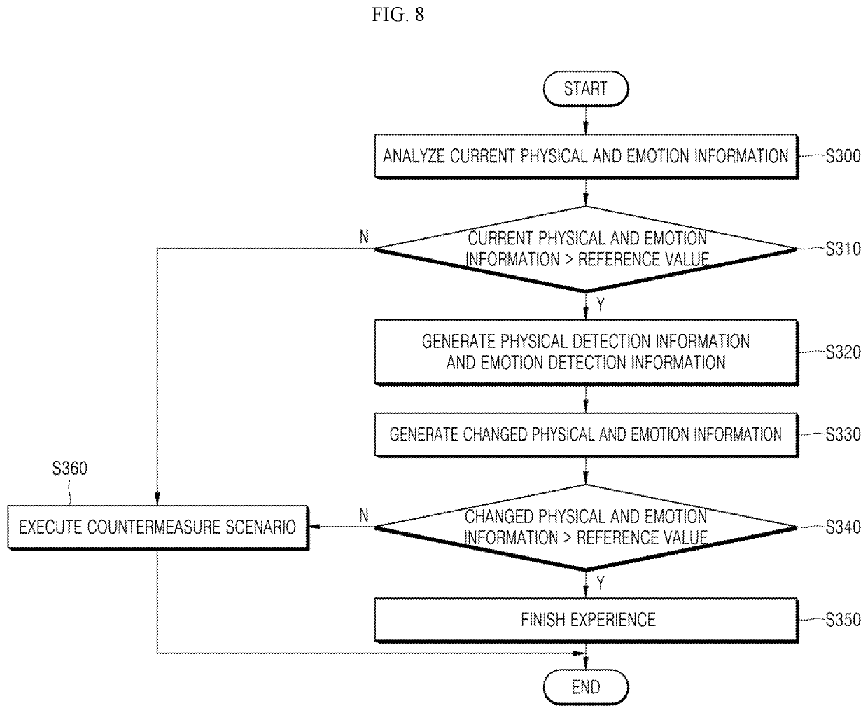

The system for providing a customized safety education and experience service for disasters on the basis of VR according to an exemplary embodiment of the present invention having the above-described configuration operates as follows. FIG. 5 is a flowchart illustrating a method of providing a customized safety education and experience service for disasters on the basis of VR according to an exemplary embodiment of the present invention, and FIG. 6 is a flowchart illustrating an operation of generating a customized experience scenario shown in FIG. 5. FIG. 7 is a flowchart illustrating an operation of executing an experience service shown in FIG. 5 according to an exemplary embodiment of the present invention, and FIG. 8 is a flowchart illustrating the operation of executing an experience service shown in FIG. 5 according to another exemplary embodiment of the present invention.

First, as shown in FIG. 5, the management server 20 may generate standard data on the basis of big data (S10).

For example, the management server 20 may generate the standard data for a VR-based disaster experience service by repeatedly learning big data using a deep learning technique or a machine learning technique.

Subsequently, the management server 20 may receive experience request information of the experiencer A from the experience terminal 10 (S12).

Specifically, the management server 20 may receive objective information and subjective information from the experiencer A.

For example, basic information, bio-information, and medical information may be determined as objective information, and the additional information, the emotion information, and other information may be determined as subjective information.

Subsequently, when the experience request information is received from the experience terminal 10, the management server 20 may generate a customized experience scenario on the basis of the standard data (S14).

Specifically, when the experience request information is received from the experiencer A who enters a virtual experience space, the management server 20 may analyze the experience request information on the basis of the standard data and generate a customized experience scenario for each disaster situation.

According to the present embodiment, customized experience scenarios may include situational countermeasure scenarios which include a countermeasure scenario for a case in which there is a disaster during execution of the experience service, a countermeasure scenario for a case in which there is no disaster, a countermeasure scenario for a case in which the experiencer A is in great danger after a disaster occurs, and a countermeasure scenario for a case in which the experiencer A is not in great danger after a disaster occurs. However, situational countermeasure scenarios are not limited thereto and may include a countermeasure scenario for an emotion change of the experiencer A when the experiencer A is in great danger after a disaster occurs.

As shown in FIG. 6, the management server 20 may receive surrounding information from a customized VR video (S100) and then determine that there is a disaster when the received surrounding information is larger than a preset reference value (S110).

Specifically, the management server 20 may analyze the surrounding information of the experiencer A which is input in real time in the virtual experience space, and when surrounding cold or warm feeling information included in the surrounding information is larger than the preset reference value, may determine that there is a disaster.

For example, when fire, smoke, and hot air around the experiencer A last three minutes or more within a radius of 100 mm in accordance with the surrounding information received in the virtual experience space, the thermoelectric element 120 may receive the surrounding cold or warm feeling information, and the management server 20 may determine that there is a disaster of a fire.

According to an exemplary embodiment, the management server 20 may analyze the surrounding information of the experiencer A which is input in real time in the virtual experience space, and when the surrounding information is larger than the reference value, may determine that there is a disaster and generate a countermeasure scenario for a case in which there is a disaster.

Subsequently, when it is determined that there is a disaster due to a value of the surrounding information larger than the reference value (S110), the management server 20 may receive current location information of the experiencer A from a customized VR video (S120).

Subsequently, the management server 20 may analyze the surrounding information and the current location information on the basis of the standard data. When the experiencer A is determined to be in great danger (S130), the management server 20 may receive current physical and emotion information of the experiencer A (S140) and generate a countermeasure scenario by analyzing the current physical and emotion information (S150).

Specifically, when a disaster occurs, the management server 20 may determine whether the experiencer A is in danger by analyzing surrounding information and current location information. When it is determined that the experiencer A is in danger, the management server 20 may compare and analyze basic physical and emotion information of the experiencer A extracted from the experience request information and the current physical and emotion information received from the experiencer A and generate a countermeasure scenario for a case in which the experiencer A is in great danger.

According to an exemplary embodiment, the management server 20 may analyze the surrounding information and the current location information on the basis of the standard data, and when it is determined that the experiencer A is in great danger, may generate a countermeasure scenario for a case in which the experiencer A is in great danger.

Meanwhile, after it is determined that there is a disaster due to the value of the surrounding information larger than the reference value, the management server 20 may analyze the surrounding information and the current location information on the basis of the standard data and determine that the experiencer A is not in great danger (S130). In this case, the management server 20 may generate a countermeasure scenario for a case in which the experiencer A is not in great danger.

Alternatively, the management server 20 may analyze the surrounding information of the experiencer A input in real time in the virtual experience space, and when the surrounding information is smaller than the reference value, may determine that there is no disaster (S110) and generate a countermeasure scenario for a case in which there is no disaster.

After that, when the experiencer A enters the virtual experience space, the management server 20 may execute the experience service using the customized experience scenario (S16).

Specifically, as shown in FIG. 7, the management server 20 may output a customized VR video corresponding to the customized experience scenario to the experiencer A (S200). A situational countermeasure scenario may be executed in accordance with the virtual experience space and the customized VR video.

Subsequently, the management server 20 may receive surrounding information detected in accordance with the customized VR video corresponding to the virtual experience space (S210), and when the received surrounding information is larger than the preset reference value, may determine that there is a disaster (S220).

For example, when the surrounding cold or warm feeling information is larger than the preset reference value on the basis of the surrounding information, the management server 20 may determine that there is a disaster.

Subsequently, when it is determined that there is a disaster, the management server 20 may receive current location information of the experiencer A (S230) and determine whether the experiencer A is in danger by analyzing the surrounding information and the current location information (S240).

Specifically, when it is determined that there is a disaster and current cold or warm feeling information is determined to be larger than the preset reference value on the basis of the surrounding information and the current location information, the management server 20 may determine that the experiencer A is in danger.

Subsequently, the management server 20 may execute the countermeasure scenario in accordance with the result of determining whether the experiencer A is in danger (S250).

According to an exemplary embodiment, the management server 20 may determine whether the experiencer A is in danger, and when it is determined that the experiencer A is in danger, may finish the experience in accordance with the countermeasure scenario.

Alternatively, when the surrounding cold or warm feeling information is smaller than the preset reference value on the basis of the surrounding information (S220), the management server 20 may determine that there is no disaster and execute a scenario corresponding to this case.

For example, the management server 20 may determine that there is no disaster around the experiencer A and continuously output the customized VR video corresponding to the customized experience scenario.

Also, when the current cold or warm feeling information is smaller than the preset reference value on the basis of the surrounding information and the current location information (S240), the management server 20 may determine that the experiencer A is not in danger and execute a scenario corresponding to this case.

According to an exemplary embodiment, as shown in FIG. 8, the management server 20 may determine whether the experiencer A is in danger using the surrounding information and the current location information which are analyzed on the basis of the customized experience scenario, and when it is determined that the experiencer A is in danger, may receive and analyze current physical and emotion information of the experiencer A (S300).

Subsequently, when the current physical and emotion information is determined to be larger than the preset reference value on the basis of the standard data (S310), the management server 20 may generate bio-detection information by detecting a physical change and generate emotion detection information by detecting an emotion change (S320).

Subsequently, the management server 20 may generate changed physical and emotion information by comparing basic physical information with the bio-detection information and comparing basic emotion information with the emotion detection information (S330).

Subsequently, the management server 20 may analyze the changed physical and emotion information on the basis of the standard data, and when the changed physical and emotion information is larger than the reference value (S340), may determine that the experiencer A undergoes a large emotion change and physical change and execute a countermeasure scenario for finishing the experience (S350).

According to an exemplary embodiment, the management server 20 may execute a countermeasure scenario for dealing with an unexpected situation corresponding to a disaster during execution of the experience service.

Meanwhile, the management server 20 may analyze the changed physical and emotion information on the basis of the standard data, and when the changed physical and emotion information is smaller than the reference value (S340), may determine that the experiencer A undergoes a small emotion change and physical change and execute a countermeasure scenario for continuing the experience service (S360).

After finishing the experience of the experiencer A, the management server 20 may generate result data corresponding to result information by assessing the quality of the experience on the basis of a result and a score of the experience service (S18).

After receiving the experience service corresponding to the experience request information, the experience terminal 10 may generate a feedback signal for the service (S20).

Here, the feedback signal may include information, such as satisfaction, accuracy, reliability, etc., on the experience service.

Subsequently, the management server 20 may generate a feedback control signal for improving the service in accordance with the feedback signal received from the experience terminal 10 (S22).

Subsequently, the management server 20 may update the standard data in real time using the customized experience scenario or the result data (S24).

According to an exemplary embodiment, the management server 20 may update the standard data in real time in accordance with at least one of the received feedback signal, the feedback control signal, and the countermeasure scenario.

Finally, the manager terminal 30 may monitor the service in real time (S26).

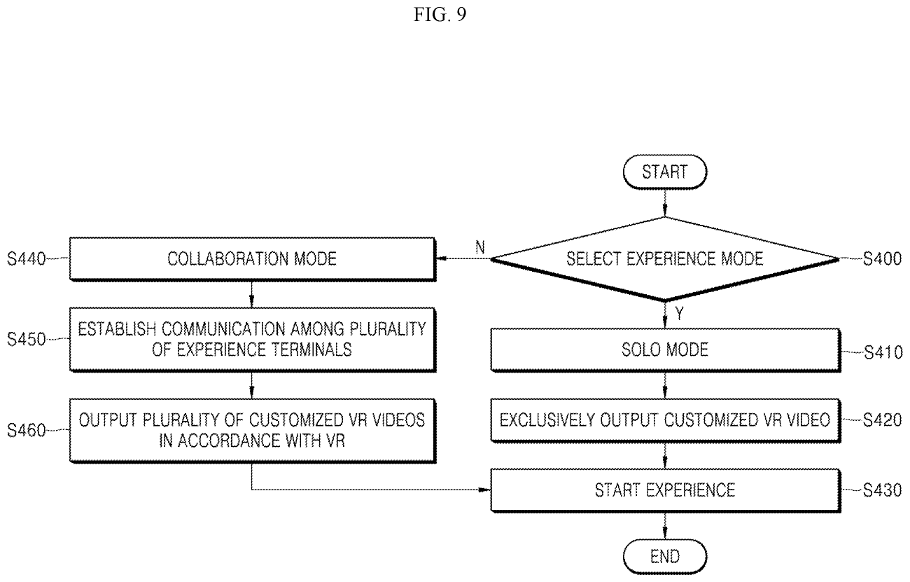

FIG. 9 is a flowchart illustrating a method of providing a customized safety education and experience service for disasters on the basis of VR according to another exemplary embodiment of the present invention.

Referring to FIG. 9, the management server 20 may select an experience mode of a solo mode or a collaboration mode (S400).

Subsequently, when the experience mode is the solo mode in which only one person experiences VR (S410), the management server 20 may exclusively output a customized VR video corresponding to a customized experience scenario to start the experience (S420 and S430).

Alternatively, when the experience mode is not the solo mode but the collaboration mode in which at least two people experience VR (S440), the management server 20 may transmit a customized VR video in accordance with VR (S450 and S460).

Specifically, in the collaboration mode, the management server 20 may establish a communication link in advance so that experience terminals 10 carried by a plurality of experiencers A who enter a virtual experience space communicate with each other, and then may transmit a customized VR video in accordance with VR so that the same customized VR video is output to the experience terminals 10.

According to the present invention, an experiencer experiences a disaster through a direct stimulus on the skin using a thermoelectric element for adjusting the feeling of cold or warm on the basis of VR and thus can be further immersed in the VR through a feeling on the experiencer's skin.

According to the present invention, it is possible to provide situation-specific and individual-specific customized safety education services in consideration of types of disasters and conditions of experiencers in the case of receiving safety education on the basis of VR.

According to the present invention, an experiencer experiences various types of disastrous situations and unexpected situations in advance through a virtual experience and thus can accurately recognize the dangerousness of disastrous situations and properly deal with various actual disasters.

According to the present invention, an experiencer receives safety education in accordance with his or her physical and mental conditions in preparation for disasters. Accordingly, it is possible to accurately recognize the dangerousness of disastrous situations which may occur in the future and properly deal with various actual disasters.

According to the present invention, various forms of VR related to disasters, such as natural disasters, safety-related accidents, industrial accidents, etc., are provided to people of all types and ages using a device that may be put on their bodies. Accordingly, it is possible to educate an experiencer to understand the power and dangerousness of each disaster and also to experience a countermeasure and a way of dealing with a disaster.

Effects of the present invention are not limited to those described above, and other effects which have not been described can be clearly understood by those of ordinary skill in the art from the above descriptions.

Operations of a method or algorithm described in connection with exemplary embodiments of the present invention may be directly implemented by hardware, implemented as a software module which is executed by hardware, or implemented by a combination thereof. The software module may be on a random access memory (RAM), a read only memory (ROM), an erasable programmable ROM (EPROM), an electrically erasable programmable ROM (EEPROM), a flash memory, a hard disk, a detachable disk, a compact disc (CD)-ROM, or any form of computer-readable recording medium well known in the technical field to which the present invention pertains.

Although exemplary embodiments of the present invention have been described above with reference to the accompanying drawings, those skilled in the technical field to which the present invention pertains should understand that the present invention can be implemented in other specific forms without changing the technical spirit or essential characteristics thereof. Therefore, the above-described exemplary embodiments should be understood to be exemplary but not limiting in all aspects.

What is claimed is:

1. A method of providing a customized safety education and experience service for disasters on the basis of virtual reality (VR) by a server, the method comprising:
receiving experience request information from an experiencer who enters a virtual experience space;
generating a customized experience scenario using the experience request information; and
executing an experience service using the customized experience scenario, wherein the executing of the experience service comprises:
receiving surrounding information detected in accordance with a customized VR video corresponding to the virtual experience space;
analyzing the surrounding information to determine whether there is a disaster;
analyzing the surrounding information and current location information in accordance with a result of determining whether there is a disaster to determine whether the experiencer is in danger;
receiving current physical and emotion information of the experiencer in accordance with a result of determining whether the experiencer is in danger;
generating changed physical and emotion information by analyzing basic physical and emotion information and the current physical and emotion information; and
executing a countermeasure scenario generated in accordance with the changed physical and emotion information on the basis of standard data.

2. The method of claim 1, wherein the determination of whether there is a disaster comprises:
determining that there is a disaster when surrounding cold or warm feeling information is larger than a preset reference value on the basis of the surrounding information; and
determining that there is no disaster when the surrounding cold or warm feeling information is smaller than the preset reference value on the basis of the surrounding information.

3. The method of claim 2, wherein the determination of whether the experiencer is in danger comprises:
determining that the experiencer is in danger when it is determined that there is a disaster and current cold or warm feeling information is larger than the preset reference value on the basis of the surrounding information and the current location information; and
determining that the experiencer is not in danger when it is determined that the current cold or warm feeling information is smaller than the preset reference value on the basis of the surrounding information and the current location information.

4. The method of claim 3, wherein the generating of the changed physical and emotion information comprises:
when the current physical and emotion information is larger than a preset reference value, detecting a physical change to generate bio-detection information and detecting an emotion change to generate emotion detection information; and
comparing basic physical information in the basic physical and emotion information with the bio-detection information and comparing basic emotion information in the basic physical and emotion information with the emotion detection information to generate the changed physical and emotion information.

5. The method of claim 4, wherein the executing of the countermeasure scenario comprises:
analyzing the changed physical and emotion information on the basis of the standard data, and when the analyzed changed physical and emotion information is larger than the preset reference value, determining that the experiencer undergoes a great emotion change and physical change and generating a countermeasure scenario for finishing the experience service; or
analyzing the changed physical and emotion information on the basis of the standard data, and when the analyzed changed physical and emotion information is smaller than the preset reference value, determining that the experiencer undergoes a small emotion change and physical change and generating a countermeasure scenario for continuing the experience service.

6. The method of claim 1, wherein, depending on a situation, the countermeasure scenario includes at least one of a countermeasure scenario for a case in which there is a disaster during the executing of the experience service, a countermeasure scenario for a case in which there is no disaster, a countermeasure scenario for a case in which the experiencer is in great danger after a disaster occurs, a countermeasure scenario for a case in which the experiencer is not in great danger after a disaster occurs, and a countermeasure scenario for an emotion change of the experiencer when the experiencer is in great danger after a disaster occurs.

7. The method of claim 1, further comprising, after the experience service is finished, assessing quality of the experience on the basis of a result and a score of the experience service to generate result data, and generating a customized experience scenario using the experience request information of the experiencer on the basis of the result data.

8. The method of claim 1, further comprising, before the executing of the experience service, transmitting the customized VR video in accordance with VR in a collaboration mode in which at least two people experience VR rather than a solo mode.

9. A computer program stored in a computer-readable recording medium to perform the method of any one of claim 1 in combination with a computer which is hardware.
